(19) United States
(12) Patent Application Publication
(10) Pub. No.: US 2003/0220587 A1
(43) Pub. Date: Nov. 27, 2003

(54) Medical device

(75) Inventor: Kirk D. Swenson, North Caldwell, NJ (US)

Correspondence Address: Kirk M. Miles, Webb Ziesenheim Logsdon Orkin & Hanson, P.C., 700 Koppers Building, 436 Seventh Avenue, Pittsburgh, PA, 15219-1818, US

(73) Assignee: Becton Dickinson and Company

(21) Appl. No.: 10/443,384

(22) Filed: May 22, 2003

Related U.S. Application Data
(60) Provisional application No. 60/382,745, filed on May 23, 2002.

(51) Int. Cl. A61B 5/00

(52) U.S. Cl. 600/576

(57) ABSTRACT

A shieldable blood collection set includes a needle cannula and a tip guard axially movable along the needle cannula through a guard drive. The tip guard is axially movable along the needle cannula from a proximal position substantially adjacent a proximal end of the needle cannula, to a distal position in which the tip guard protectively surrounds a puncture tip at the distal end of the needle cannula. The guard drive interconnects the needle cannula and the tip guard through a hub which includes a rigid fin and a tether. The rigid fin is pivotally connected to the hub and extends dorsally from the hub, and the tether interconnects the end of the fin with the tip guard. Pivotal movement of the fin causes the tether to extend to move the tip guard from the proximal position to the distal position for shielding of the needle.

### CROSS REFERENCE TO RELATED APPLICATIONS

This application claims the benefit of U.S. Provisional Application No. 60/382,745 filed on May 23, 2002 which is incorporated by reference in its entirety.

### BACKGROUND OF THE INVENTION

1. Field of the Invention

The present invention relates to blood collection sets for safe and convenient handling of needles. More particularly, the present invention relates to a low cost disposable blood collection set including a needle assembly having a safety shield.

2. Description of Related Art

Disposable medical devices having piercing elements are typically used for administering a medication or withdrawing a fluid, such as blood collecting needles, fluid handling needles and assemblies thereof Current medical practice requires that the fluid containers and needle assemblies used in such systems be inexpensive and readily disposable. Consequently, existing blood collection systems, for example, typically employ some form of durable, reusable holder on which detachable and disposable needles and fluid collection tubes may be mounted. A blood collection system of this nature can be assembled prior to use and then disassembled after usage. Thus, these blood collection systems allow repeated use of the relatively expensive holder upon replacement of the relatively inexpensive needle and/or fluid collection tube. In addition to reducing the cost of collecting blood specimens, these blood collection systems also help minimize the production of hazardous medical waste.

A blood collection set or intravenous (IV) infusion set typically includes a needle cannula having a proximal end, a pointed distal end and a lumen extending therebetween. The proximal end of the needle cannula is securely mounted in a plastic hub with a central passage that communicates with the lumen through the needle cannula. A thin flexible thermoplastic tube is connected to the hub and communicates with the lumen of the needle cannula. The end of the plastic tube remote from the needle cannula may include a fixture for connecting the needle cannula to a blood collection tube or some other receptacle. The specific construction of the fixture will depend upon the characteristics of the receptacle to which the fixture will be connected.

In order to reduce the risk of incurring an accidental needle-stick wound, protection of used needle tips becomes important. With concern about infection and transmission of diseases, methods and devices to enclose the used disposable needle have become very important and in great demand. For example, needle assemblies commonly employ a safety shield that can be moved into shielding engagement with a used needle cannula without risking an accidental needle stick.

Some needle shields are referred to as tip guards, and include a small rigid guard that can be telescoped along the length of a needle cannula and extended over the puncture tip of the needle for protection. Such conventional tip guard may include some form of tether for limiting the travel of the tip guard to the length of the needle cannula. Additionally, such conventional tip guard typically includes a structure that lockingly engages over the tip of the used needle cannula to prevent a re-exposure of the needle. The structure for preventing re-exposure may include a metallic spring clip or a transverse wall integrally formed with one end of the tip guard. Needle assemblies including such tip guards, however, typically include extensive mechanics for positioning of the tip guard, resulting in complex arrangements which are costly to manufacture and assemble. Also, operation of the tip guard can involve substantial manipulation by the user to extend the tip guard to a shielding position.

PCT International Publication No. WO 98/57689 discloses a shield mechanism for catheter introducer needles which includes a hinged arm mechanism for moving a tip guard along a needle to a shielding position. The hinged arm mechanism disclosed in this publication, however, includes a number of interengaging hinge mechanisms which cause the hinged arm to collapse upon itself in one position and to entirely extend or unfold to move the tip guard along the needle to the shielding position. As such, during use, the hinge arm entirely unfolds from the collapsed condition, thereby eliminating the hinged arm from the profile of the device. Moreover, the device includes complicated interengaging structure which can be difficult to manufacture and assemble.

While the above described devices provide for effective shielding of used needles, a need exists for a needle assembly for use with a blood collection set which achieves secure and effective shielding of a used needle tip and which is simple and inexpensive to manufacture and easy to operate.

### SUMMARY OF THE INVENTION

The present invention is directed to a shieldable needle device, particularly useful in connection with a blood collection set. The needle device includes a needle cannula having opposed proximal and distal ends and a lumen extending therebetween. The needle device further includes a hub having a proximal end, a distal end, and a passage extending between the ends. The proximal end of the needle cannula is securely mounted in the passage of the hub. A flexible tube may be mounted to the proximal end of the hub, such that the passage through the tube communicates with the lumen of the needle cannula. A fixture may be mounted to the end of the tube remote from the hub. The fixture enables the needle cannula and the tube to be placed in communication with an appropriate receptacle, such as a blood collection tube.

The needle device further includes a shield assembly, such as an end cap having a blocking surface that is slidably telescoped on the needle cannula for axial movement from a proximal position substantially adjacent the hub at the proximal end of the needle cannula to a distal position where the end cap protectively surrounds the distal end of the needle cannula. Additionally, the end cap may be configured to prevent proximal movement after the blocking surface of the end cap has advanced sufficiently in a distal direction to protectively enclose the distal tip of the needle assembly.

The end cap may be in the form of a tip guard, which may include a tip guard housing formed from a plastic material, and a protective clip, such as a metallic spring clip mounted to the housing. The clip is biased against the needle cannula when the tip guard is in the proximal position and is resiliently moved over the distal end and distal tip of the needle cannula when the tip guard is in the distal position, thereby preventing piercing by the tip of the needle.

The needle device further includes a guard drive for moving the tip guard from the proximal position to the distal position. The guard drive includes a rigid fin having a first end pivotally connected to the hub and an opposed second end extending dorsally from the hub. The fin is pivotally movable between a proximal position and a distal position toward the distal end of the needle cannula. The guard drive further includes a tether extending between the second end of the fin and the tip guard. The fin and the tether are adapted for corresponding movement such that pivotal movement of the fin with respect to the hub from the proximal position to the distal position causes the tether to extend toward the distal end of the needle cannula for movement of the tip guard from the proximal position to the distal position.

The needle device may include a latch for preventing movement of the tip guard from the proximal position to the distal position. The hub may include a pair of wings extending laterally from opposing sides of the hub. Also, the fin may include a pair of concave surfaces on opposing sides thereof. The tether may be a flexible material, and is desirably connected to the fin through a hinge, such as a living hinge providing for corresponding movement between the fin and the tether. In particularly desirable embodiments, the fin is integrally formed with the tether and the hub. As such, the needle device is passive, with automatic shielding of the needle tip occurring upon release of the latch, thereby permitting movement of the tip guard from the proximal position to the distal position.

### DESCRIPTION OF THE DRAWINGS

FIG. 1 is a perspective view of a blood collection set in accordance with the present invention including a packaging cover thereon;

FIG. 2 is an exploded perspective view of the shieldable needle assembly of the blood collection set of FIG. 1;

FIG. 3 is a top plan view of the shieldable needle assembly in a retracted position;

FIG. 4 is a cross-sectional view taken along line 4-4 of FIG. 3;

FIG. 5 is a top plan view of the shieldable needle assembly in an extended shielded position;

FIG. 6 is a cross-sectional view taken along line 6-6 of FIG. 5;

FIG. 7 is a cross-sectional view of a shieldable needle assembly in an alternative embodiment shown in a retracted position;

FIG. 8 is a cross-sectional view of the shieldable needle assembly of FIG. 8 shown in an extended shielded position;

FIG. 9 is a perspective view of a guard drive and hub shown in a further alternative embodiment;

FIG. 10 is a cross-sectional view of a shieldable needle assembly in an alternative embodiment including the guard drive of FIG. 9 shown in a retracted position;

FIG. 11 is a cross-sectional view of the shieldable needle assembly in an alternative embodiment including the guard drive of FIG. 9 shown in an extended shielded position;

FIG. 12 is a perspective view of a guard drive and hub shown in yet a further alternative embodiment including an alternative latching mechanism; and

FIGS. 13A, 13B and 13C are front views of the alternative mechanism of FIG. 12 showing the latch positioning during use of the shieldable needle assembly.

### DETAILED DESCRIPTION

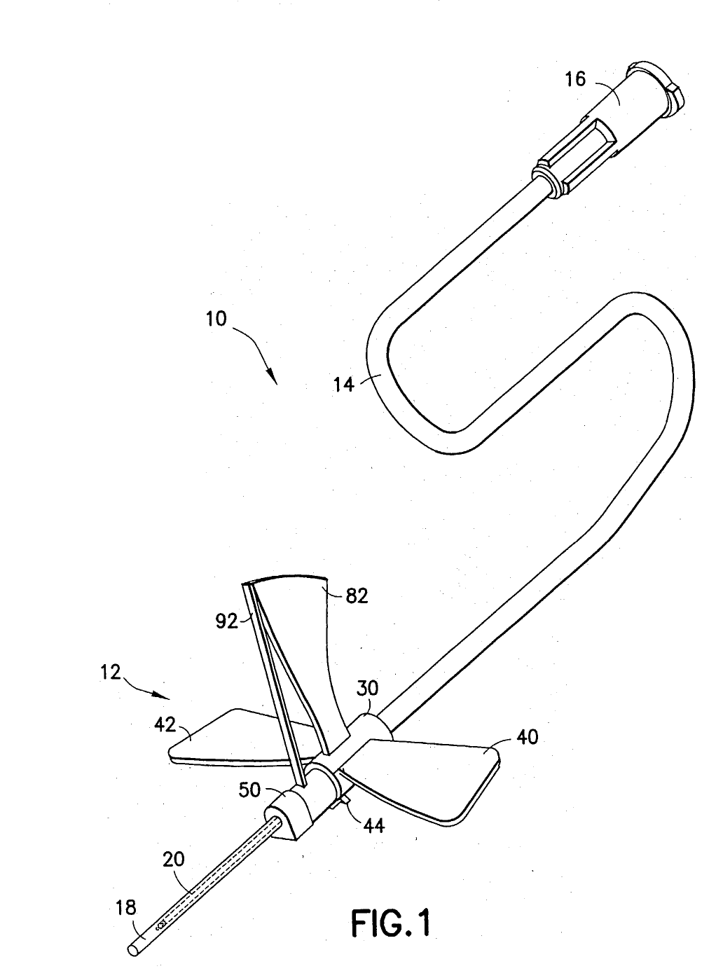

Referring to the drawings in which like reference characters refer to like parts throughout the several views thereof, FIG. 1 illustrates a blood collection set in accordance with the present invention and the related features. The present invention is generally described in terms of a blood collection set, and encompasses such a blood collection set, as well as a shieldable needle assembly for use in such a blood collection set.

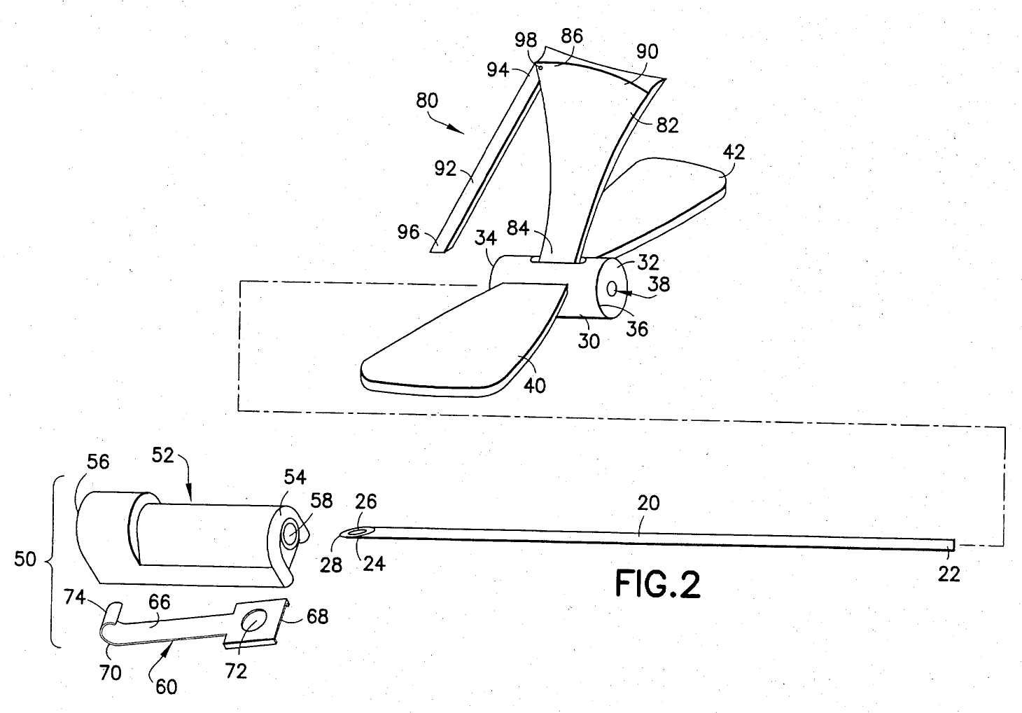

As shown in FIG. 1, blood collection set 10 includes a shieldable needle device 12, a flexible tube 14 extending from needle device 12, a fixture 16 mounted to tube 14, and a packaging cover 18 removably mounted to portions of needle device 12 opposite tube 14, such as through a frictional engagement. Shieldable needle device 12 of blood collection set 10 is shown in detail in FIGS. 2-4, and includes a needle cannula 20, a hub 30, a tip guard assembly 50 and a guard drive 80, for moving the tip guard assembly.

Needle cannula 20 includes a proximal end 22 and an opposing distal end 24, with lumen 26 extending through needle cannula 20 from proximal end 22 to distal end 24. Distal end 24 of needle cannula 20 is beveled to define a sharp puncture tip 28, such as an intravenous puncture tip. Puncture tip 28 is provided for insertion into a patient's blood vessel, such as a vein, and is therefore designed to provide ease of insertion and minimal discomfort during venipuncture.

Needle assembly 12 further includes hub 30. Hub 30 is a unitary structure, desirably molded from a thermoplastic material. Hub 30 includes a proximal end 32, a distal end 34, and is defined by a rigid tubular wall 36 extending from proximal end 32 to distal end 34. Tubular wall 36 is characterized by an internal passage 38 extending therethrough from proximal end 32 to distal end 34 of hub 30. A recess 48 extends within a top portion of tubular wall 36 of hub 30.

Hub 30 further includes a pair of stabilizers in the form of flexible wings 40 and 42 extending laterally from tubular wall 36 at opposing sides thereof. Wings 40 and 42 provide hub 30, and needle assembly 12, as a butterfly-type wing assembly, assistance in positioning and placement of needle assembly 12 and blood collection set 10 during a blood collection procedure, and providing a surface for securing needle device 12 to the skin of a patient, such as, through taping wings 40 and 42 to the patient's skin.

Needle cannula 20 is positioned within internal passage 38 of hub 30, and extends from distal end 34 of hub 30. Desirably, needle cannula 20 and hub 30 are separate parts which are fixedly attached and secured through an appropriate medical grade adhesive or the like.

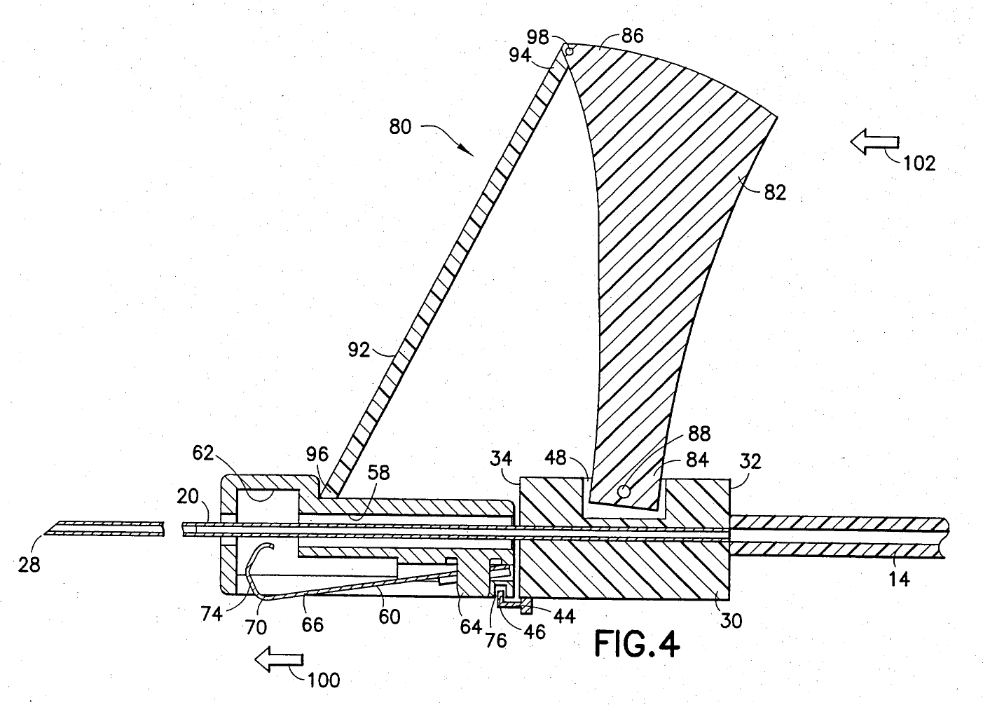

Needle assembly 12 further includes tip guard assembly 50, which extends co-axially about needle cannula 20 and is movable along needle cannula 20 between a first proximal position adjacent hub 30, and a second distal position adjacent puncture tip 28, as will be described in more detail herein. Tip guard assembly 50 includes a housing 52 and a protective clip 60. Housing 52 is a unitary structure, desirably molded from a thermoplastic material, including a proximal end 54, a distal end 56, and an internal passage 58 extending between the ends. Portions of internal passage 58 adjacent distal end 56 define an enlarged clip receptacle 62, as shown in FIG. 4. A clip mounting post 64 extends downwardly from housing 52 at a location near proximal end 54 of housing 52.

Clip 60 is unitarily stamped and formed from a resiliently deflectable metallic material. Clip 60 includes a planar spring leg 66 with a proximal end 68 and an opposed distal end 70. A mounting aperture 72 extends through spring leg 66 at a location near proximal end 68. Mounting aperture 72 has a diameter approximately equal to or slightly less than the diameter of mounting post 64 of housing 52. As such, mounting post 64 can be forced through mounting aperture 72 when the axis of mounting post 64 and the axis of mounting aperture 72 are substantially collinear. A lock out leg 74 extends angularly from distal end 70 of spring leg 66. Lock out leg 74 is bent back toward proximal end 68 of clip 60. The bends in lock out leg 74 enable secure protective engagement with puncture tip 28 of needle cannula 20 and further enable smooth axial sliding movement of tip guard assembly 50 along needle cannula 20, as described in further detail herein.

Hub 30 and tip guard assembly 50 are interconnected through guard drive 80. Guard drive 80 provides for axial movement of tip guard assembly 50 along needle cannula 20 from a first proximal position adjacent hub 30 to a second distal position adjacent puncture tip 28, as will be described in more detail herein.

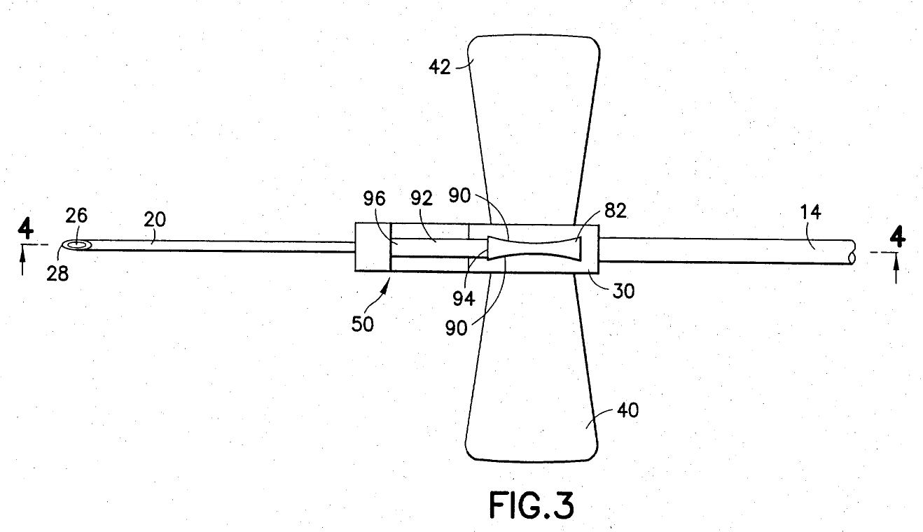

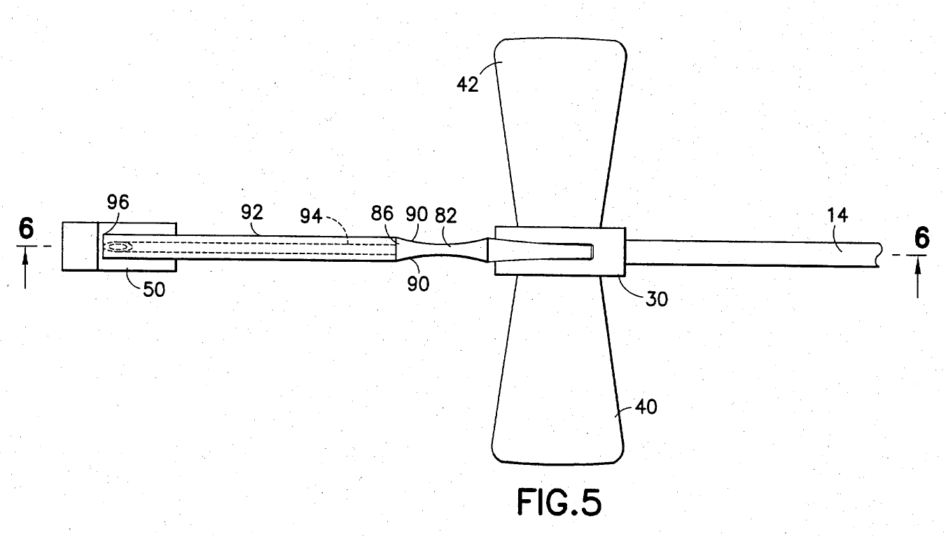

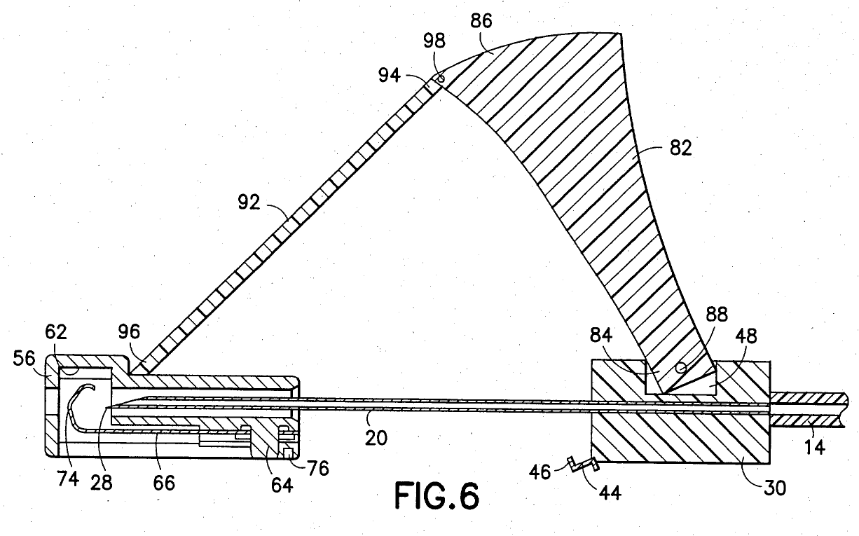

Guard drive 80 includes a fin 82 extending dorsally from a top surface of hub 30. Fin 82 is a generally rigid structure including a first portion 84 and a second portion 86. First portion 84 of fin 82 is pivotally connected to hub 30 for establishing pivotal movement of fin 82 with respect to hub 30. Such pivotal movement may be established, for example, by providing first portion 84 within recess 48 at the top surface of tubular wall 36 of hub 30, and providing a pivot 88 extending from hub 30 and through first portion 84 of fin 82 in a direction perpendicular to the general axis of needle device 12. In this manner, fin 82 may be pivoted about an axis defined by pivot 88, thereby causing fin 82 to move in a planar direction with respect to the general axis of needle device 12 from a proximal retracted position in which fin 82 extends dorsally from hub 30 as shown in FIGS. 3 and 4, to a distal extended position in which fin 82 remains dorsally extending from hub 30, as shown in FIGS. 5 and 6.

As indicated, fin 82 extends from a top surface of hub 30 in a dorsal manner, that is, in a plane with the general axis of needle device 12, providing fin 82 with opposing sides facing laterally away from a plane of symmetry. Such opposing sides desirably include concave surfaces 90. Concave surfaces 90 are designed and configured for engagement with a thumb and index finger of a user during placement of blood collection set 10, as will be discussed in further detail herein.

Guard drive 80 further includes a tether 92 extending between tip guard assembly 50 and second portion 86 of fin 82. Tether 92 is desirably a resilient flexible material capable of bending and/or extending when a force is applied thereto. Tether 92 includes proximal portion 94 and distal portion 96. Distal portion 96 of tether 92 may be fixedly attached to tip guard assembly 50, such as through the use of an adhesive. Proximal portion 94 of tether 92 is connected to second portion 86 of fin 82, such as through hinge 98. Hinge 98 provides for corresponding movement between fin 82 and tether 92, as will be discussed in more detail herein.

Since proximal portion 94 of tether 92 is connected to second portion 86 of fin 82, pivotal movement of fin 82 about pivot 88 from the proximal position to the distal position results in corresponding movement of tether 92. In particular, movement of fin 82 against hinge 98 exerts a biasing force against tether 92, thereby forcing tether 92 to extend in a direction toward distal end 24 of needle cannula 20. Moreover, since distal portion 96 of tether 92 is fixedly attached to tip guard assembly 50 and since tip guard assembly 50 is axially movable along needle cannula 20, such biasing force against tether 92 causes tip guard assembly 50 to axially move in the direction of arrow 100 away from hub 30 and toward distal end 24 of needle cannula 20, where tip guard assembly 50 can effectively shield puncture tip 28.

Tip guard assembly 50 moves axially along needle cannula 20 toward distal end 24 during movement of guard drive 80 through corresponding movement between fin 82 and tether 92. By providing hinge 98 therebetween as a living hinge, fin 82 and tether 92 can be effectively biased against each other. As such, movement of fin 82 from the proximal position to the distal position exerts a biasing force against tether 92, thereby forcing distal portion 96 of tether 92 toward distal end 24 of needle cannula 20. Tether 92 may also be a flexible material. As such, tether 92 may itself act as a means for storing energy to extend tether 92 toward distal end 24 of needle cannula 20 upon corresponding movement between fin 82 and tether 92, thereby propelling tip guard assembly 50 from the proximal position to the distal position.

Needle device 12 may also be provided with means for retaining tip guard assembly 50 in the proximal position and preventing movement of tip guard assembly 50 from the proximal position adjacent hub 30 to the distal position adjacent puncture tip 28. For example, hub 30 may include a latch 44 extending from a lower portion of tubular wall 36 adjacent distal end 34, extending in a direction toward distal end 24 of needle cannula 20. Latch 44 is provided for releasable engagement with tip guard assembly 50, such as through frictional interfitting engagement between latch 44 and tip guard assembly 50. Such interfitting engagement prevents movement of tip guard assembly 50 from the proximal position to the distal position when latch 44 is engaged with recess 76. Desirably, latch 44 is a hinged element which includes a protrusion 46 for extending within recess 76 within the bottom of housing 52 of tip guard assembly 50, and which is capable of hinged movement between a first position in which protrusion 46 extends within recess 76 and is therefore in frictional engagement with tip guard assembly 50, and a second position in which protrusion 46 is out of recess 76 and therefore out of frictional engagement with tip guard assembly 50. As such, latch 44 can be released from interfitting engagement with recess 76 of tip guard assembly 50, thereby permitting movement of tip guard assembly 50 from the proximal position to the distal position.

It is also contemplated that hub 30 may include a latch (not shown) for preventing pivotal movement of fin 82 within recess 48 of hub 30, in addition to or instead of latch 44 extending between hub 30 and tip guard assembly 50.

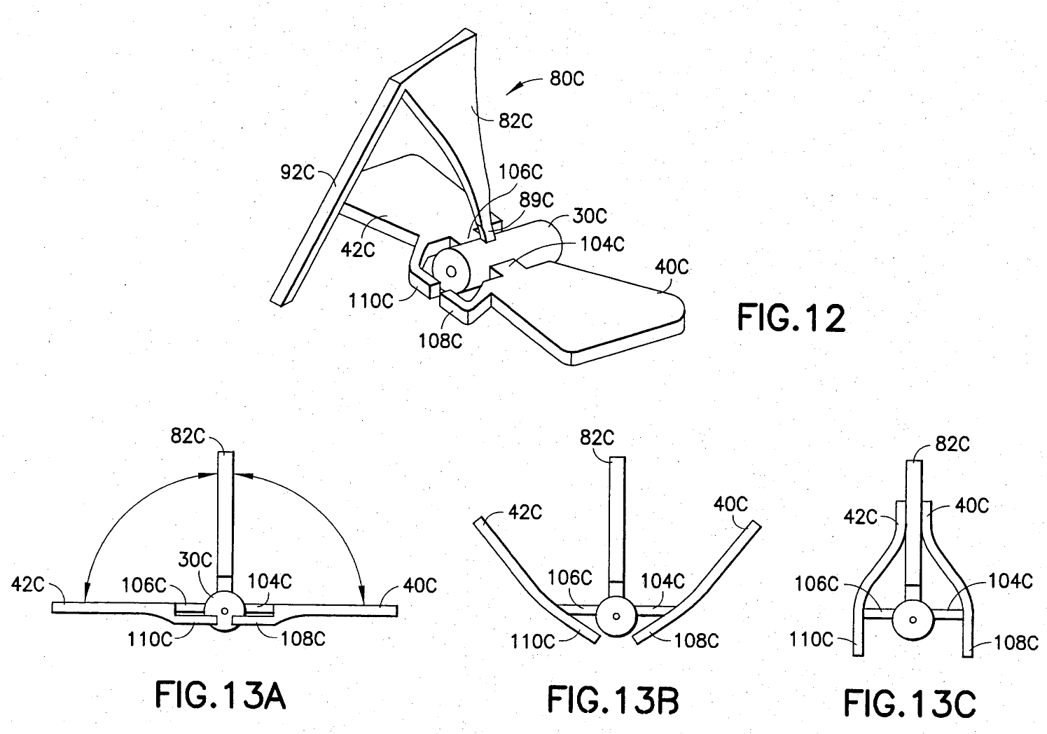

In particularly desirable embodiments, latch 44 is in corresponding engagement with at least one of wings 40 and/or 42. For example, as noted above, hub 30 may include wings 40 and 42 extending laterally from opposing sides of tubular wall 36. Wings 40 and 42 assist in positioning and placement of needle assembly 12 and blood collection set 10 during a blood collection procedure, and are adapted to lie flat against the surface of a patient's skin during a blood collection procedure. As such, wings 40 and 42 may be constructed of a flexible material such that at least one, and possibly both, of wings 40 and 42 can be bent toward each other and brought together between the fingers of a user to assist in positioning and placement of needle assembly 12 during venipuncture. By providing latch 44 in corresponding engagement with at least one of wings 40 and 42, such bending of wings 40 and/or 42 will cause latch 44 to be released from interfitting engagement with recess 76 of tip guard assembly 50, thereby permitting movement of tip guard assembly 50 from the proximal position to the distal position. Such corresponding engagement may be provided, for example, through a connection member extending between latch 44 and at least one of wings 40 and 42, such that bending of wings 40 and 42 will cause latch 44 to release, by way of the connection member. One particular embodiment for such a release mechanism is shown in detail with reference to FIGS. 12-13C, as described herein.

Guard drive 80 may require active exertion of force thereon in order to effect movement of tip guard assembly 50 from the proximal position to the distal position in the direction of arrow 100. For example, once latch 44 is released from engagement with tip guard assembly 50, a force may be exerted by the user on fin 82 in the direction of arrow 102. Such force causes fin 82 to pivotally move with respect to hub 30 about pivot 88 in a direction toward distal end 34 of hub 30 and toward distal end 24 of needle cannula 20, i.e., between a proximal position and a distal position. Such pivotal movement causes tether 92 to extend toward distal end 24 of needle cannula 20. Since tip guard assembly 50 is fixedly attached to tether 92, such extension also causes movement of tip guard assembly 50 from the proximal position adjacent hub 30 to the distal position adjacent puncture tip 28.

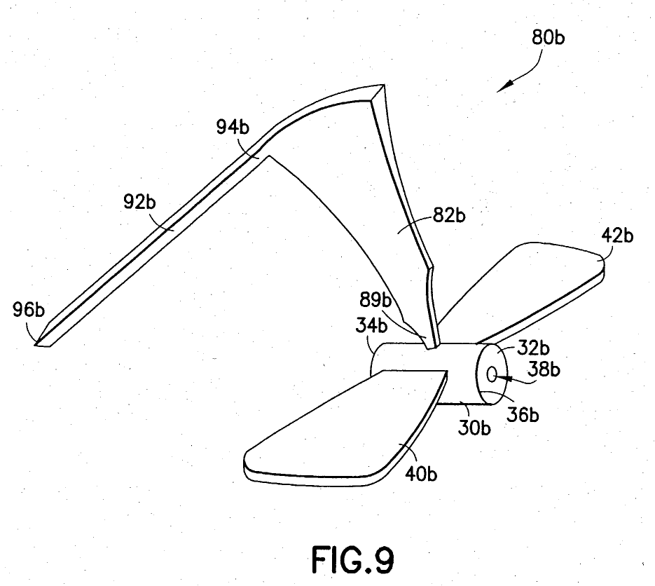

In particularly desirable embodiments, needle device 12 is passively shieldable in that it is capable of achieving secure shielding of puncture tip 28 automatically upon release of latch 44. To achieve such passive shielding, guard drive 80 may include means for storing energy for movement of fin 82 and/or tether 92 in a direction toward distal end 24 of needle cannula 20. For example, hinge 98 may be a torsion spring capable of exerting a biasing force upon release of engagement between hub 30 and tip guard assembly 50, such as through latch 44, to extend tether 92 toward distal end 24 of needle cannula 20, thereby propelling tip guard assembly 50 from the proximal position to the distal position. Alternatively, pivot 88 may include a torsion spring capable of exerting a biasing force upon release of engagement between hub 30 and tip guard assembly 50 to move fin 82 from the proximal position to the distal position, thereby extending tether 92 toward distal end 24 of needle cannula 20 and propelling tip guard assembly 50 from the proximal position to the distal position. In a further embodiment discussed herein with reference to FIGS. 9-11, such passive shielding can be achieved, for example, by providing guard drive 80 as a unitary structure including fin 82 integrally formed with tether 92 and hub 30, and with living hinges established therebetween which are capable of exacting biasing force to extending tether 92 and to thereby propel the tip guard assembly 50 from the proximal position to the distal position.

Tip guard assembly 50 is assembled by forcing mounting post 64 of tip guard housing 52 through mounting aperture 72 of clip 60. Spring leg 66 of clip 60 is then urged downwardly or away from internal passage 58 through tip guard housing 52. Distal end 22 of needle cannula 20 is then passed through internal passage 38 of hub 30, and urged into internal passage 58 at proximal end 54 of tip guard housing 52. The downward deflection of spring leg 66 enables distal end 24 of needle cannula 20 to be passed entirely through tip guard housing 52. Spring leg 66 can be released after puncture tip 28 of needle cannula 20 passes entirely through tip guard housing 52. Thus, the end of lock out leg 74 will be biased against and slide along needle cannula 20. Tip guard assembly 50 then is slid proximally along needle cannula 20 into a position adjacent hub 30. Packaging cover 18 is then urged over puncture tip 28 and urged proximally over needle cannula 20, with puncture tip 28 safely maintained and disposed within packaging cover 18.

Blood collection set 10 can be packaged substantially in the condition shown in FIG. 1. Prior to use, blood collection set 10 is removed from its package. Fixture 16 then may be connected to an appropriate receptacle for providing fluid communication with lumen 26 through needle cannula 20.

In use, blood collection set 10 is provided with needle device 12 assembled and including flexible tube 14 extending from needle device 12 and connected to fixture 16. After removing blood collection set 10 from its package, it can be assembled with other appropriate medical equipment for use. For example, a non-patient needle assembly and a needle holder may be connected to blood collection set 10 through fixture 16.

To prepare for use of blood collection set 10, the user grasps blood collection set 10 at needle device 12, placing a thumb and forefinger between one concave surface 90 of fin 82 and one of wings 40 or 42 of hub 30, with fin 82 maintained between the user's fingers. Alternatively, both wings 40 and 42 can be flexed or bent toward each other between a user's thumb and forefinger with fin 82 trapped therebetween. Fin 82 may be of sufficient length to extend dorsally beyond wings 40 and 42 when wings 40 and 42 are flexed or bent together, thereby providing a further surface for grasping between the user's thumb and forefinger. Packaging cover 18 is then grasped and urged distally to disengage from needle cannula 20, thereby exposing puncture tip 28 of needle cannula 20.

The medical practitioner can then urge puncture tip 28 at distal end 24 of needle cannula 20 into a targeted blood vessel of a patient, while guard drive 80 is maintained between thumb and forefinger to assist in controlled entry by the medical practitioner. During such positioning, at least one of wings 40 and 42 is bent inwardly toward the other and toward fin 82 between the user's fingers. Such bending causes latch 44 to disengage from engagement with tip guard assembly 50. Alternately, latch 44 may be manually released by the user's finger. In embodiments incorporating a torsion spring or other biasing means as described above, tip guard assembly 50 is maintained in the proximal position due to the grip by the user's fingers between one of wings 40 or 42 and the fin 82 at concave surface 90, even though latch 44 is released. As such, fin 82 is prevented from movement from the proximal position to the distal position due to the user's grasp against concave surface 90.

After the targeted blood vessel has been accessed, the medical practitioner can release the grip on guard drive 80. Once the user releases the device, fin 82 is free to pivotally move from the proximal position to the distal position, due to the bias exerted between tether 92 and fin 82 through hinge 98. Such movement causes tether 92 to extend, thereby propelling tip guard assembly 50 distally along needle cannula 20 in an axial direction of arrow 100, with tip guard assembly 50 sliding or gliding along needle cannula 20 toward distal end 24. Distal movement of tip guard assembly 50 will terminate when proximal end 54 of tip guard housing 52 contacts the skin of the patient near the puncture site.

Upon completion of the procedure, such as when all desired samples have been drawn, needle cannula 20 is withdrawn from the patient. This removal of needle cannula 20 from the patient will permit further extension of tether 92 and a corresponding distal movement of tip guard assembly 50 in an axial direction of arrow 100. After tip guard assembly 50 is moved along needle cannula 20 to the distal end 24, lockout leg 74 of clip 60 will pass distally beyond puncture tip 28 of needle cannula 20. The inherent resiliency of spring leg 66 of clip 60 will urge lockout leg 74 over puncture tip 28 of needle cannula 20. Thus, a return movement of tip guard assembly 50 is prevented. Furthermore, guard drive 80 has an overall dimension that will prevent movement of tip guard assembly 50 distally beyond needle cannula 20. Hence, puncture tip 28 of needle cannula 20 is safely shielded. Blood collection set 10 may then be appropriately discarded.

Since fin 82 of guard drive 80 extends dorsally from hub 30, fin 82 can act as a handle portion during insertion, withdrawal and disposal of needle device 12. In particular, even after activation of the shielding feature in order to propel tip guard assembly 50 to the distal position shielding needle cannula 20, fin 82 is maintained in a dorsal position, albeit at a slightly forward or distal position. Since fin 82 still extends dorsally and is a rigid structure, fin 82 can be used to grip needle device 12 after removal from the patient, and can act as a handle portion for carrying blood collection set 10 at a position remote from the used needle tip of cannula 20.

It is noted that while activation of the safety feature may be automatically accomplished upon release of latch 44, providing needle device 12 with a passively shielding feature, it is also contemplated that activation of the shielding feature may require a specific force exerted by the user in a direction of arrow 102, as discussed above. In such situations, guard drive 80 can be activated while puncture tip 28 is within the patient's blood vessel, thereby axially moving tip guard assembly 50, axially along needle cannula 20, or may be activated after puncture tip 28 is removed from the patient's blood vessel.

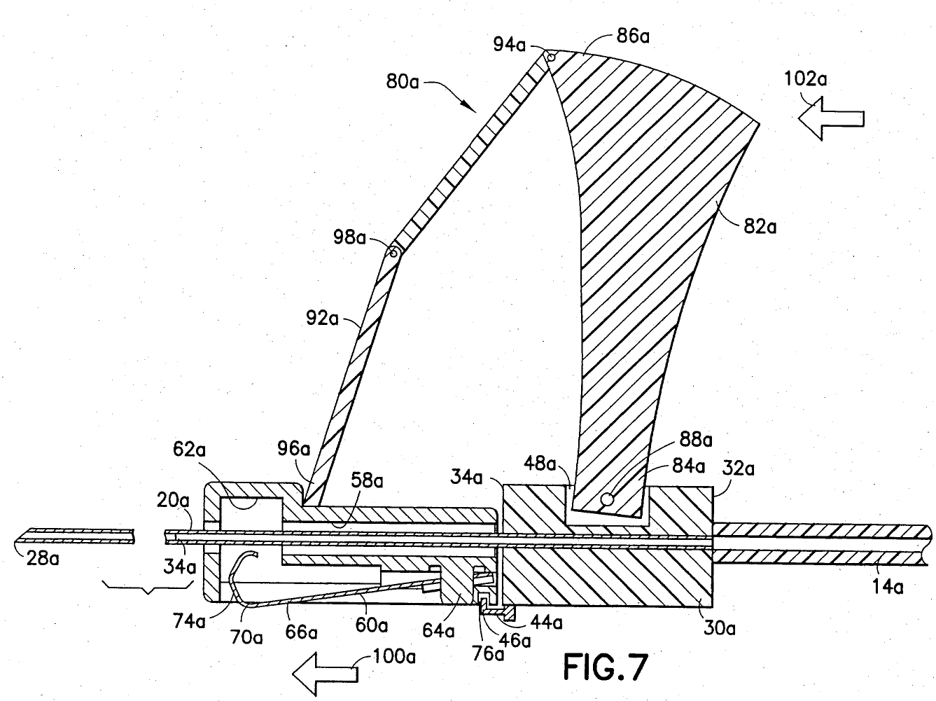

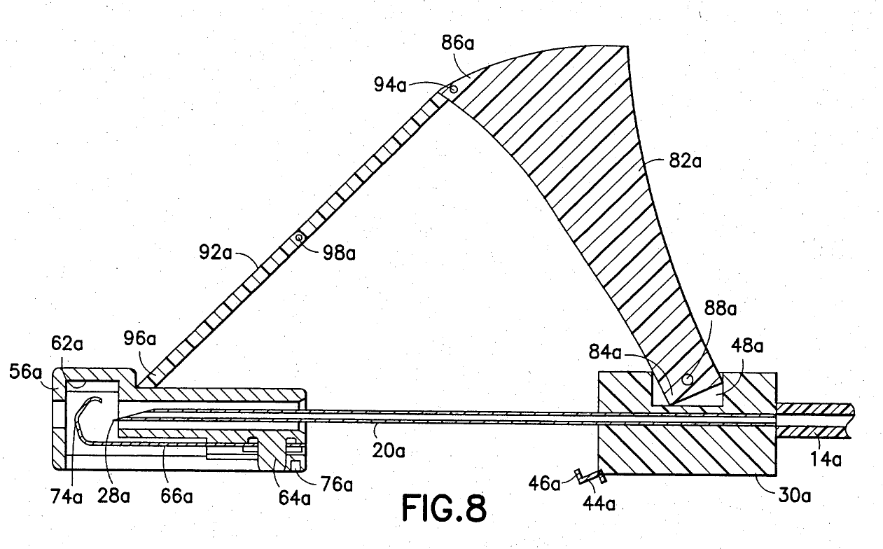

FIGS. 7-13 depict further embodiments of the present invention that include many components which are substantially identical to the components of FIGS. 1-6. Accordingly, similar components performing similar functions will be numbered identically to those components of FIGS. 1-6, except that a suffix “a” will be used to identify those similar components in FIGS. 7 and 8, a suffix “b” will be used to identify those similar components in FIGS. 9-11, and a suffix “c” will be used to identify those similar components in FIGS. 12 and 13A-C.

In a further embodiment depicted in FIGS. 7 and 8, the tether 92a includes two sections that meet at a hinge 98a. In particular, guard drive 80a includes tether 92a including proximal portion 94a and distal portion 96a. Proximal portion 94a is fixedly adhered to fin 82a at second portion 86a of fin 82a, desirably in a flexing joint. Distal portion 96a is fixedly adhered to tip guard assembly 50a as in the embodiment described above with reference to FIGS. 1-6. Proximal portion may be a rigid material, and is preferably constructed of a similar material as fin 82a. In particularly preferred embodiments, proximal portion 94a is integrally formed with fin 82a as an extension thereof with the juncture between proximal portion 94a and fin 82a acting as a living hinge. Distal portion 96a may be a bendable or flexible material, capable of extending.

Proximal portion 94a and distal portion 96a are interconnected through hinge 98a. Hinge 98a may be a living hinge, but is desirably a torsion spring capable of exerting a biasing force upon release of engagement between hub 30a and tip guard assembly 50a to extend distal portion 96a of tether 92a toward distal end 24a of needle cannula 20a, thereby propelling tip guard assembly 50a from the proximal position to the distal position. As such, needle device 12a is provided with a passive shielding feature.

In a further embodiment depicted in FIGS. 9-11, guard drive 80b is integrally formed with hub 30b and tether 92b, forming a one-piece unitary structure. In particular, guard drive 80b includes a fin 82b extending dorsally from and pivotal with respect to a top surface of hub 30b in a similar manner as in the embodiment described with respect to FIGS. 1-6. Pivotal movement of guard drive 80b, however, is established through the specific structure of hub 30b and fin 82b which creates a living hinge 89b for pivotal movement therebetween. Desirably, the structure of fin 82b is cut away on the opposing sides thereof to form a narrow portion at first end 84b at the point of connection with hub 30b. This narrowing portion allows for flexibility between fin 82b and hub 30b, with the wall portion of fin 82b at this narrowing portion acting as a living hinge for pivoting movement of fin 82b.

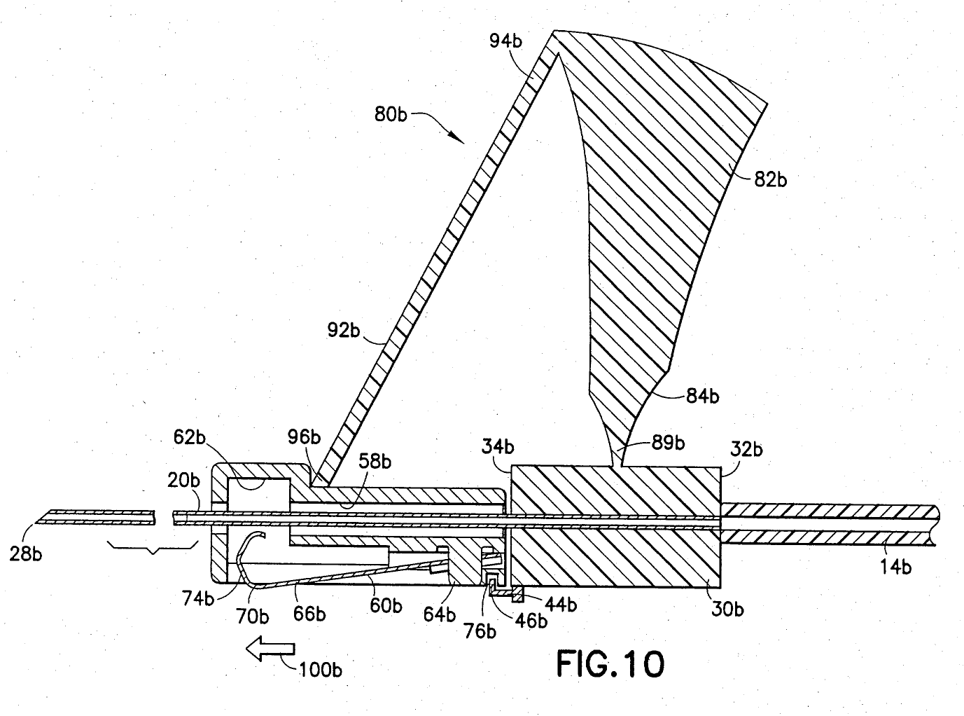

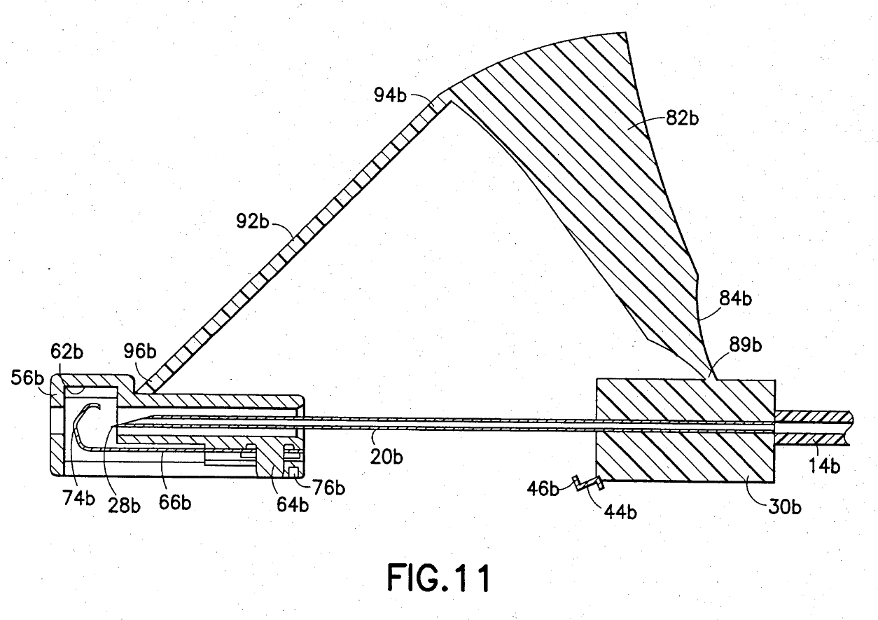

As noted, guard drive 80b is desirably integrally formed with tether 92b and hub 30b. Desirably, guard drive 80b is an integral structure which is in a relaxed state with fin 82b in a distal position and with tether 92b extended toward the distal position, as shown in FIGS. 9 and 11. As such, the natural tendency for guard drive 80b is to return to this relaxed state with fin 82b in a distal position and with tether 92b extended toward the distal position. In such an embodiment, the device may be assembled and packaged with guard drive 80b retained in the proximal position as shown in FIG. 10, such that guard drive 80b is biased against its natural relaxed state. In this manner, guard drive 80b is effectively “primed” for operation to propel the tip guard to a distal position encompassing puncture tip 28b of needle cannula 20b. Guard drive 80b may be retained in this proximal position against its natural bias through any mechanism, such as through friction established by a packaging cover covering needle cannula 20b, or through a latch 44b as described in connection with the embodiment described above.

It is further contemplated that guard drive 80b is in a relaxed stated during packaging. Prior to use, guard drive 80b can be pivoted to the proximal position and held in such a position between wings 40b and 42b held between a user's finger and thumb such that guard drive 80b is biased against its natural relaxed stated and is primed for operation. Upon release, guard drive 80b returns to its natural unbiased stated, and therefore propels the tip guard to a distal position encompassing puncture tip 28b of needle cannula 20b.

As described above, in embodiments incorporating biasing means for providing a passively shielding feature, such as by providing hinge 98a as a torsion spring or by providing guard drive 80b with a living hinge 89b which is biased against a relaxed state, passive shielding of the needle cannula is automatically achieved merely by removing needle cannula from the patient. In some instances, however, the needle device may be dropped or knocked from the hand of the medical practitioner either before venipuncture or during a medical procedure. The passive shielding described above will commence automatically when the needle device is dropped or knocked from the medical practitioner's hand. Thus, the automatic shielding may be triggered by the intentional or unintentional release of the fin by the medical practitioner.

Moreover, a medical practitioner does not always enter the targeted blood vessel during the first venipuncture attempt. However, a medical practitioner typically retains a close grip on the needle device until the targeted blood vessel has been entered. In this instance, the continued gripping between the fin and the wings will prevent the needle from shielding until the targeted blood vessel has been punctured. The second attempt at accessing a targeted blood vessel generally is a very low risk procedure in which the practitioner's hand is spaced considerably from the puncture tip of the needle cannula. Thus, the blood collection set according to the present invention does not involve the inconvenience of having to use a new blood collection set following each unsuccessful venipuncture attempt.

As noted above, FIGS. 12-13C depict a further embodiment of the present invention which includes an alternate latch mechanism for retaining the tip guard assembly in the proximal position adjacent hub 30c and preventing movement of the tip guard assembly until use. In particular, the embodiment shown in FIGS. 12-13C demonstrates a latch mechanism which is in corresponding engagement with at least one, and desirably both of the flexible wings extending laterally from the hub, as described above in connection with the embodiment shown in FIGS. 1-6.

In the embodiment of FIGS. 12-13C, wings 40c and 42c extend laterally from opposing sides of hub 30c and are interconnected therewith through rigid struts 104c and 106c, respectively. Rigid struts 104c and 106c are desirably constructed of the same material as hub 30c, and are rigid members which are not meant to flex, or are meant to flex only slightly during bending of wings 40c and 42c. As such, rigid struts 104c and 106c act in effect as fulcroms for flexing of wings 40c and 42c during use. Alternatively, wings 40c and 42c may include a rigid portion at the point of attachment with hub 30c, thereby eliminating the need for any such rigid struts 104c and 106c.

Wings 40c and 42c include latches 108c and 110c, respectively, which extend from the forward or distal edges thereof radially inward toward the distal end 34c of hub 30c. Latches 108c and 110c are configured for releasable interfitting engagement with a recessed portion of the tip guard assembly, such as recess 76 of tip guard assembly 50, described above and depicted in FIG. 4. Desirably, such a recessed portion is in the form of an annular ring extending about at least a proximal portion of the housing of the tip guard assembly, such that latches 108c and 110c extend within such an annular ring when wings 40c and 42c are in their natural state extending laterally from opposing sides of hub 30c in a planar manner. In this manner, latches 108c and 110c are in interference engagement with the tip guard assembly and therefore retain the tip guard assembly at a proximal position adjacent hub 30c.

During use, wings 40c and 42c are bent inwardly toward each other and toward fin 82c, to assist in positioning and placement of the needle assembly during a procedure, as described above. During such bending, rigid struts 104c and 106c act as fulcroms for bending of wings 40c and 42c, as shown in sequence in FIGS. 13A-13C. In this manner, latches 108c and 110c in turn are moved or pivoted out of interference engagement with the recess of the tip guard assembly. In particularly desirable embodiments which incorporate a biasing means for biasing the guard drive forward toward the distal position for shielding of the needle such as the guard drive 80b including living hinge 89b described in connection with FIGS. 9-11, release of latches 108c and 110c will cause the tip guard assembly to be released from its retracted proximal position. As shown in FIG. 13C, however, when wings 40c and 42c are in a position to release latches 108c and 110c, wings 40c and 42c are bent against fin 82c of guard drive 80c, for example between a user's finger and thumb. As such, the pressure between wings 40c and 42c between a user's finger and thumb exerts a normal force between wings 40c and/or 42c and guard drive 80c, thereby retaining fin 82c in the proximal dorsal position, thus preventing movement of fin 82c to the distal dorsal position which would cause activation of guard drive 80c to propel the tip guard assembly distally forward. As shown in FIG. 13C, ideally fin 82c has a sufficient dorsal length when measured from hub 30c to allow for a dorsal portion of fin 82c to remain exposed in relation to wings 40c and 42c when the wings are moved towards adjacent alignment with fin 82c. This provides the user with the ability to hold fin 82c in its proximal dorsal position between a user's finger and thumb and have sufficient clearance to move wings 40c and 42c in and out of a position bent against fin 82c, and where a user could re-grip fin 82c along with wings 40c and 42c.

Once the needle assembly is properly placed at the intended site, the user can release the grip between wings 40c and 42c, which will release fin 82c, and in turn will release guard drive 80c for activation, at which time the tip guard assembly will move forward to a position against the patient's skin until the needle is removed from the patient, at which time the tip guard assembly with automatically and passively shield the needle tip. In this manner, the needle assembly is passively activated during a normal sequence of operation and use, in that the typical operation of bending of the wings for positioning and placement of the needle causes release of the tip guard assembly, and the same motion for bending of the wings also retains the thus released tip guard assembly in the retracted position until the needle assembly is released from the user's grip.

While the needle assembly of the present invention has been described in terms of one embodiment for use in connection with a blood collection system, it is further contemplated that the needle assembly could be used with other medical procedures, such as in conjunction with a conventional intravenous infusion set, which are well known in the art for use with needle assemblies.

While the present invention is satisfied by embodiments in many different forms, there is shown in the drawings and described herein in detail, the preferred embodiments of the invention, with the understanding that the present disclosure is to be considered as exemplary of the principles of the invention and is not intended to limit the invention to the embodiments illustrated. Various other embodiments will be apparent to and readily made by those skilled in the art without departing from the scope and spirit of the invention. The scope of the invention will be measured by the appended claims and their equivalents.

What is claimed:

1. A shieldable needle device comprising:
a needle cannula having opposed proximal and distal ends;
a hub mounted to said proximal end of said needle cannula;
a tip guard axially movable along said needle cannula from a proximal position substantially adjacent said hub to a distal position where said tip guard protectively surrounds said distal end of said needle cannula; and
a guard drive for moving said tip guard from said proximal position to said distal position, said guard drive comprising a rigid fin having a first end pivotally connected to said hub and an opposed second end extending dorsally from said hub and movable from a proximal dorsal position to a distal dorsal position, said guard drive further comprising a tether extending between said second end of said fin and said tip guard, said fin and said tether adapted for corresponding movement such that pivotal movement of said fin with respect to said hub from said proximal dorsal position to said distal dorsal position causes said tether to extend toward said distal end of said needle cannula for movement of said tip guard from said proximal position to said distal position.

2. A needle device as in claim 1, further comprising a latch for preventing movement of said tip guard from said proximal position to said distal position.

3. A needle device as in claim 1, wherein said hub includes a pair of wings extending laterally from opposing sides of said hub.

4. A needle device as in claim 1, wherein said fin includes a pair of concave surfaces on opposing sides thereof.

5. A needle device as in claim 1, wherein said tether is connected to said second end of said fin through a hinge, said hinge providing said corresponding movement between said fin and said tether.

6. A needle device as in claim 1, wherein said fin is integrally formed with said hub and said tether, thereby establishing a first living hinge between said first end of said fin and said hub and a second living hinge between said second end of said fin and said tether.

7. A needle device as in claim 6, wherein said guard drive is in a biased state when said fin is in said proximal dorsal position.

8. A needle device as in claim 1, wherein said tip guard comprises a tip guard housing formed from a plastic material, a metallic spring clip being mounted to said housing, said spring clip being biased against said needle cannula when said tip guard is in said proximal position and being resiliently moved over said distal end of said needle cannula when said tip guard is in said distal position.

9. A needle device as in claim 1, wherein said hub is adapted for connection to a flexible tube of a blood collection set.

10. A shieldable needle device comprising:
a needle cannula having opposed proximal and distal ends;
a hub mounted to said proximal end of said needle cannula, said hub including a rigid fin having a first end pivotally connected to said hub and an opposed second end extending dorsally from said hub, said fin pivotally movable between a dorsally extending proximal position and a dorsally extending distal position toward said distal end of said needle cannula;
a tip guard axially movable along said needle cannula from a proximal position substantially adjacent said hub to a distal position where said tip guard protectively surrounds said distal end of said needle cannula; and
a resilient flexible tether connected with said second end of said fin and extending between said fin and said tip guard,
wherein pivotal movement of said fin from said dorsally extending proximal position to said dorsally extending distal position causes said tether to extend toward said distal end of said needle cannula for movement of said tip guard from said proximal position to said distal position.

11. A needle device as in claim 10, further comprising a latch for preventing movement of said tip guard from said proximal position to said distal position.

12. A needle device as in claim 10, wherein said hub further includes a pair of wings extending laterally from opposing sides of said hub.

13. A shieldable blood collection set comprising:
a flexible tube having opposed first and second ends, said first end of said flexible tube being adapted for connection to a blood collection receptacle;
a hub mounted to said second end of said flexible tube;
a needle cannula having a proximal end connected to said hub, a distal end projecting from said hub and a lumen in fluid communication with said flexible tube and said fixture;
a tip guard axially movable along said needle cannula from a proximal position substantially adjacent said hub to a distal position surrounding said distal end of said needle cannula; and
a guard drive for moving said tip guard from said proximal position to said distal position, said guard drive comprising a rigid fin having a first end pivotally connected to said hub and an opposed second end extending dorsally from said hub and movable from a proximal dorsal position to a distal dorsal position, said guard drive further comprising a tether extending between said second end of said fin and said tip guard, said fin and said tether adapted for corresponding movement such that pivotal movement of said fin with respect to said hub from said proximal dorsal position to said distal dorsal position causes said tether to extend toward said distal end of said needle cannula for movement of said tip guard from said proximal position to said distal position.

14. A blood collection set as in claim 13, further comprising a packaging cover frictionally engaged on said needle cannula and securely surrounding said needle cannula.

15. A blood collection set as in claim 13, wherein said tip guard comprises a rigid housing having an aperture extending therethrough, said needle cannula being slidably disposed in said aperture, said tip guard further comprising a metallic clip mounted to said housing and configured for sliding engagement against said needle cannula as said tip guard moves from said proximal position toward said distal position, said metallic clip being dimensioned and disposed to protectively cover said distal end of said needle cannula when said tip guard has reached said distal position.

16. A blood collection set as in claim 13, further comprising a latch for preventing movement of said tip guard from said proximal position to said distal position.

17. A blood collection set as in claim 13, wherein said hub includes a pair of wings extending laterally from opposing sides of said hub.

18. A blood collection set as in claim 13, wherein said fin includes a pair of concave surfaces on opposing sides thereof.

19. A blood collection set as in claim 13, wherein said fin is integrally formed with said hub and said tether, thereby establishing a first living hinge between said first end of said fin and said hub and a second living hinge between said second end of said fin and said tether.

20. A blood collection set as in claim 19, wherein said guard drive is in a biased state when said fin is in said proximal dorsal position.

21. A shieldable needle device comprising:
a needle cannula having opposed proximal and distal ends;
a tip guard axially movable along said needle cannula from a proximal position substantially adjacent said proximal end of said needle cannula to a distal position where said tip guard protectively surrounds said distal end of said needle cannula; and
a hub mounted to said proximal end of said needle cannula, said hub including a pair of flexible wings extending laterally from opposing sides of said hub with at least one of said wings including a latch in engagement with said tip guard for maintaining said tip guard in said proximal position, said hub further including a guard drive comprising a rigid fin having a first end pivotally connected to said hub and an opposed second end extending dorsally from said hub and pivotal between a dorsally extending proximal position in which said guard drive is in a biased state and a dorsally extending distal position toward said distal end of said needle cannula, said guard drive further comprising a tether extending from said second end of said fin and connected with said tip guard,
wherein bending of said flexible wings toward said fin releases said latch from engagement with said tip guard and maintains said fin in said proximal position with said guard drive in a biased state, and flexing of said flexible wings away from said fin releases said fin so as to bias said guard drive toward said distal position, thereby causing said tether to extend toward said distal end of said needle cannula for movement of said tip guard from said proximal position to said distal position.

22. A needle device as in claim 21, wherein both of said pair of flexible wings includes a latch in engagement with said tip guard.

23. A needle device as in claim 21, wherein said pair of flexible wings are attached to said hub through a rigid strut.

24. A blood collection set as in claim 19, wherein said guard drive is in a biased state when said fin is in said proximal dorsal position.
